(19) United States
(12) Patent Application Publication
(10) Pub. No.: US 2007/0036714 A1
(43) Pub. Date: Feb. 15, 2007

(54) Porphyrin derivatives, methods for obtaining same, and use thereof in radioimmunotherapy

(76) Inventor: Bernard Phillippe Albert Boitrel, Melesse (FR)

Correspondence Address: YOUNG & THOMPSON, 745 SOUTH 23RD STREET, 2ND FLOOR, ARLINGTON, VA, 22202, US

(21) Appl. No.: 10/540,124

(22) PCT Filed: Dec. 18, 2003
(86) PCT No.: PCT/FR03/03794

§ 371 Date: Jun. 2, 2006

(30) Foreign Application Priority Data
Dec. 20, 2002 (FR) 02/16371
Oct. 22, 2003 (FR) 03/12341

(51) Int. Cl. A61K 51/00; C07D 487/22; C07F 5/00; A61K 31/555

(52) U.S. Cl. 424/1.11; 514/185; 514/410; 534/11; 540/145

(57) ABSTRACT

The invention concerns compounds of general formula (I), wherein: when A forms a chain with C, called A-C chain, of formula (1): —X—Y—C6H4-(CH2)n1-C(Z,W)—(CH2)n2-C6H4-Y—X—, then B forms a chain with D, the chain of above formula (1), called A-C and B-D chains located independently of each other, above (position alpha ) or below (position beta ) of the porphyrin macrocycle; or when A forms a chain with D, called A-D chain of above formula (1), then B forms a chain with C, called B-C chain of above formula (1), one of the A-D or B-C chains, being located above (position alpha) of the plane of the porphyrin macrocycle while the other A-D or B-C chain is located below (position beta )) of the porphyrin macrocycle. The invention also concerns complexes between the compounds and radioelements, and pharmaceutical compositions containing the complexes.

A subject of the present invention is novel porphyrin derivatives, processes for obtaining them, and their uses in radiotherapy or radioimmunotherapy.

The treatments currently administered in the fight against cancer mainly concern chemical drugs, and the use of sources of radiation. The main problem caused by this type of treatment is the non-specificity of these therapeutic techniques, which as a result leads to the indiscriminate damaging of healthy cells.

The discovery of monoclonal antibodies in the 1970s brought great hope to the fields of cancer diagnosis and therapy. This novel technique in fact appears to be a solution to the problems of non-specificity of antitumor agents. However, monoclonal antibodies capable of recognizing antigens on the surface of tumors do not have sufficient toxicity to destroy them. On the other hand, by combining this protein with an element capable of eliminating the diseased cells, an entity is formed which is very useful, since it is very specific and active. Thus, radioimmunotherapy combines the properties of monoclonal antibodies with that of radioactive metals. The antibody is modified during coupling with a ligand stabilizing the radioelement or directly with the radioelement (Yuanfang, L.; Chuanchu, W. Pure Appl. Chem. 1991, 63, 427).

Numerous radioelements have already been the subject of very intensive study (Yuanfang, L.; Chuanchu, W., mentioned above) in this field. Bismuth-212 or -213 is an α emitter, i.e. capable of delivering very considerable energy over a very short distance, which makes this metal very attractive for the treatment of small tumor cells. The stakes are therefore high since at present very few α emitters have useful specifications for possible use in radioimmunotherapy (Wibur, D. S. Antibody, Immunoconj.Radiopharm. 1991, 4, 85; Feinendegen, L.; McClure, J; Rad. Res. 1997, 148, 195).

The first studies relating to the coupling of a bismuth complex with a monoclonal antibody and its behaviour in vitro, were carried out in 1986 by Kozak's team (Kozak, R.; Atcher, R.; Gansow, O.; Friedman, A.; Hines, J; Waldmann, T; Proc. Natl. Acad. Sci. USA 1986, 83).

These first very encouraging investigations were carried out with the isobutylcarbonic anhydride of DTPA as a complexing agent, the formula of which is indicated below.

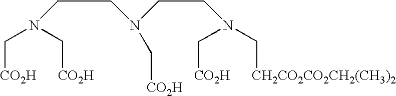

Subsequently, other types of ligands were synthesized in order to perfect the metal coordination sphere and to induce greater stability of the complexes formed. Examples are illustrated hereafter with DOTA and cyclohexylbenzyl DTPA (cyDTPA).

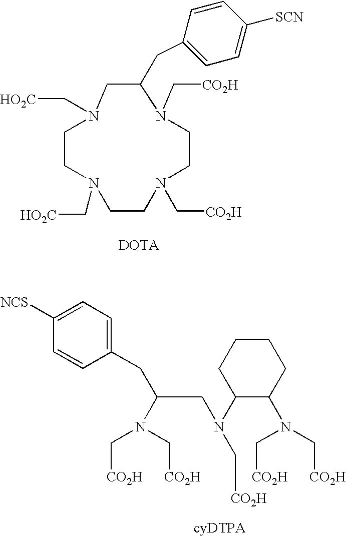

The cyDTPA represented above is at present the most promising ligand. The metallation of this ligand is very rapid (Brechbiel, M; Pippin, C.; McMurry, T; Milenic, D.; Roselli, D.; Colcher, D.; Gansow, O. J. Chem. Soc., chem. Soc. 1991, 1169), and the complex formed is relatively stable in vivo.

The choice of porphyrins as ligands is not insignificant since studies report a preferential accumulation of porphyrins in tumors (Moan, J.; Berg, K. Photochem. Photobiol. 1992, 55, 931), and their biocompatible character. Moreover, this macrocycle has unique properties due to its disc shape and its relative rigidity.

Preliminary studies, carried out by the Inventors, on so-called planar porphyrins, such as octaethylporphyrin, have shown that the metal was situated above the plane of the porphyrin. The counter-anion is important since, in the isolated complexes, the metal is linked to triflate and nitrate anions (oxygenated counter-anion). The Inventors have also attempted to metallate tetraphenylporphyrin with different bismuth salts and in particular bismuth chloride, when the reaction is carried out under argon, and followed by UV-visible spectroscopy, the start of metallation is noted but most of the starting ligand is not consumed, and the complex obtained is not stable.

The purpose of the present invention is to provide new compounds allowing the complexation of radioelements such as the a emitters, and more particularly bismuth, making it possible to form complexes with the above-mentioned radioelements which are more stable compared with the compounds of the prior art, by the presence of pre-organized handles modifying neither the geometry of the tetrapyrrolic nucleus nor its electronic properties.

A purpose of the invention is also to provide novel pharmaceutical compositions liable to be used in radiotherapy or radioimmunotherapy.

A subject of the present invention is the compounds corresponding to the following general formula (I):

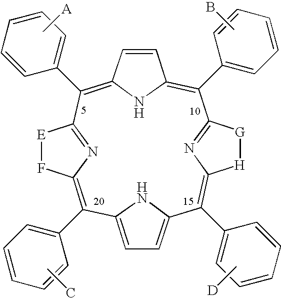

in which:

when A forms a chain with C, the so-called A-C chain, of formula (1) below:
—X—Y—C₆H₄—(CH₂)ₙ₁—U—(CH₂)ₙ₂—C₆H₄—Y—X— (1)

in which:
when X represents NH, O, CO or CH₂, Y represents respectively CO, CH₂, NH, or O,
n₁ and n₂, independently of one another represent an integer comprised between 1 and 3,
U represents a group of the C (Z, W) or N (CHRₐ—COOR_{b}) form, in which
Z represents:
   an electroattractive group such as CN, NO₂, or CO₂⁻,
   or a CH₂NR₁R₂ group, in which R₁ and R₂ represent, independently of one another, H, or a linear, branched, or cyclic alkyl group, with 1 to 8 carbon atoms, or an aryl or alkylaryl group, or a specific antibody, if appropriate linked to the CH₂N part of said group via a spacer,
   or an aryl group substituted by an SO₃R₃, SO₂R₃, p-NO₂ or o-NO₂ function, in which R₃ represents H, or a cation chosen from the alkali metals such as Na⁺, or K⁺, or R₃ represents an NR₄R₅ group in which R₄ and R₅ represent, independently of one another, a linear, branched, or cyclic alkyl group, with 1 to 8 carbon atoms, or R₃ represents a para-nitro aryl group,
W represents a CO₂⁻or COOR₆ group in which R₆ represents H or a linear, branched, or cyclic alkyl group, with 1 to 8 carbon atoms, or an aryl group, or an alcohol depopulated of electrons such as a para-nitro phenol or ortho-para-nitro phenol group,
or Z and W form in combination with the carbon atom which carries them (indicated by an arrow hereafter) a ring designated Meldrum's acid with the following formula: 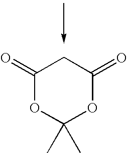
Rₐ corresponds to the definition previously given for R₁, or can also preferably represent the side chain of a natural or modified amino acid,
R_{b} corresponds to the definition previously given for R₁, then B forms a chain with D, the so-called B-D chain, of the abovementioned formula (1), said A-C, and B-D chains, being situated independently of one another, above (position α) or below (position β) the porphyrin macrocycle plane,

or when A forms a chain with D, the so-called A-D chain, of the abovementioned formula (1), then B forms a chain with C, the so-called B-C chain, of the abovementioned formula (1), one of said A-D or B-C chains being situated above (position α) the porphyrin macrocycle plane, whilst the other A-D or B-C chain, is situated below (position β) the porphyrin macrocycle plane,

E represents in combination with F, and H represents in combination with G, independently of each other, CH═CH, or CH₂—CH₂.

A more particular subject of the invention is the abovementioned compounds of formula (I), characterized in that the chain formations of formula (1) are chosen from the following:

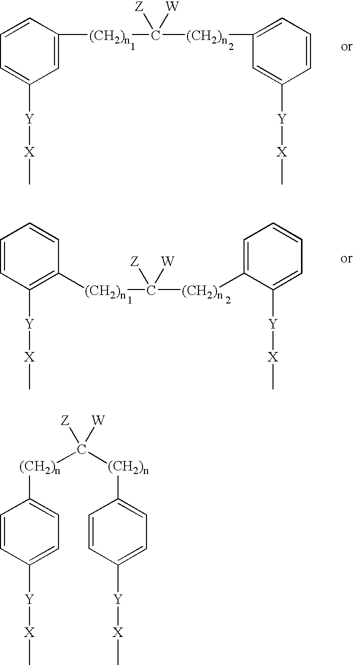

in which the Z and W groups are:

either directed towards the interior of said compounds and are situated above or below the porphyrin macrocycle plane according to whether said chain formations of formula (1) are situated respectively in α position or in β position, and are respectively designated Ziα and Wiα, or Ziβ or Wiβ,

or directed towards the exterior of said compounds, and are respectively designated Ze and We.

A more particular subject of the invention is also the compounds as defined above, characterized in that A, B, C, and D are in ortho position, as well as those characterized in that E represents in combination with F, and H represents in combination with G, CH₂-CH₂.

The invention more particularly relates to the compounds as defined above, characterized in that A forms with C, and B forms with D, chain formations of formula (1) respectively designated A-C and B-D, these two chain formations being situated in a, said compounds also being designated compounds of formula (Ia).

In this respect, a more particular subject of the invention is the abovementioned compounds of formula (Ia), characterized in that:

the A-C and B-D chain formations each comprise a Ziα group and a We group,

or the A-C chain formation comprises a Ziα group and a We group, whilst the B-D chain formation comprises a Ze group and a Wiα group,

or the A-C and B-D chain formations each comprise a Ze group and a Wiα group.

Preferred compounds of formula (Ia) are those characterized by the following formulae:

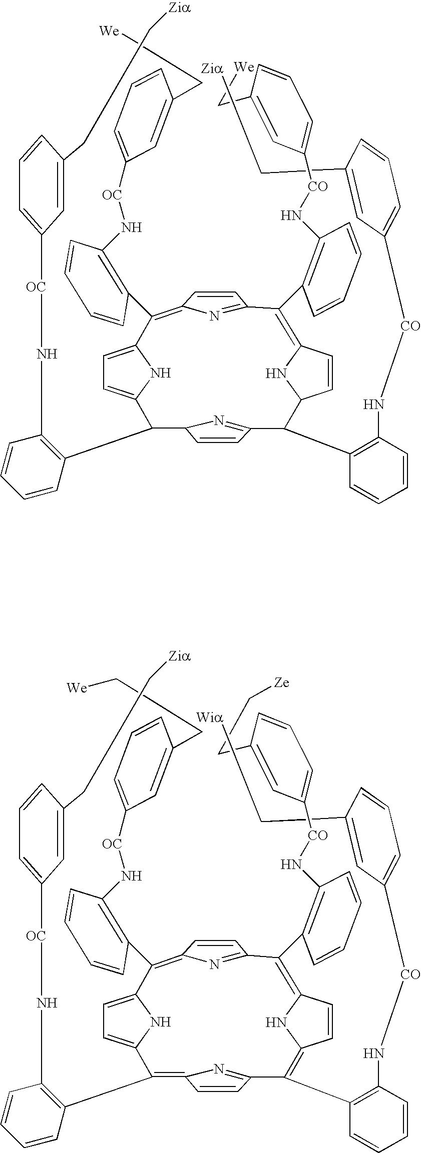

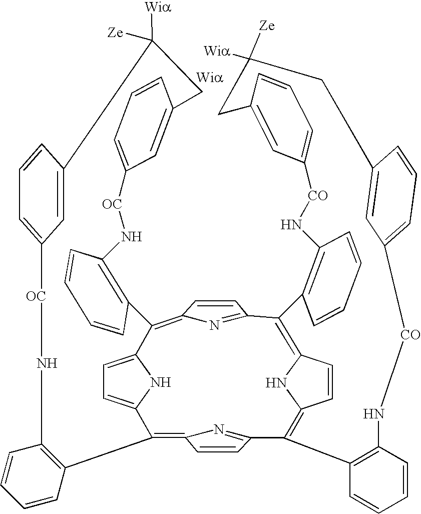

The invention also relates to the compounds of formula (I) as defined above, characterized in that A forms with C an A-C chain formation of formula (1) situated in the α position, and B forms with D, a B-D chain formation of formula (1) situated in the β position, said compounds also being designated compounds of formula (Ib).

In this respect, a more particular subject of the invention is the abovementioned compounds of formula (Ib), characterized in that:

the A-C chain formation comprises a Ziα group and a We group, whilst the B-D chain formation comprises a Ziβ group and a We group,

or the A-C chain formation comprises a Ze group and a Wiα group, whilst the B-D chain formation comprises a Ziβ group and a We group,

or the A-C chain formation comprises a Ze group and a Wiα group, whilst the B-D chain formation comprises a Ze group and a Wiβ group.

Preferred compounds of formula (Ib) are those characterized by the following formulae:

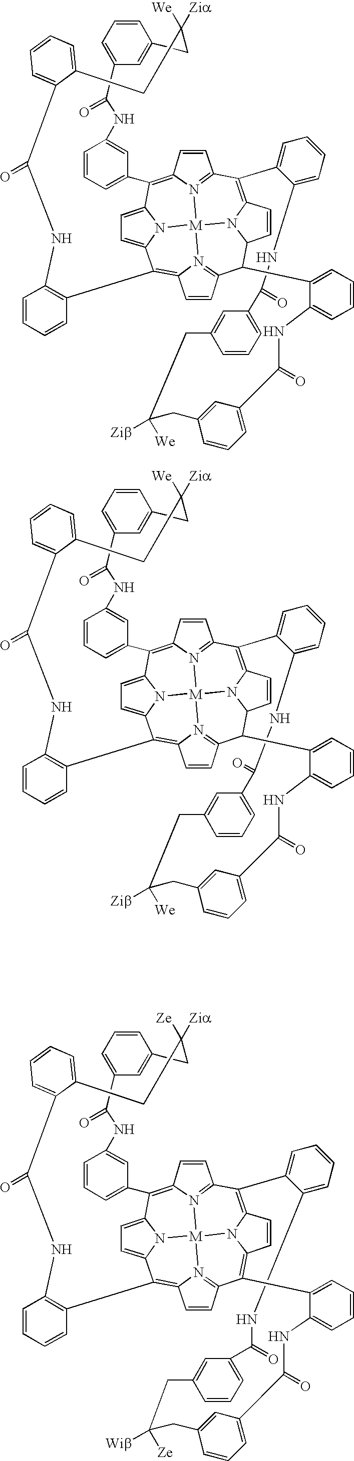

The invention also relates to the compounds of formula (I) as defined above, characterized in that A forms with D an A-D chain formation of formula (1) situated in position β, and B forms with C a B-C chain formation of formula (1) situated in position α, said compounds also being designated compounds of formula (Ic).

In this respect, a more particular subject is the abovementioned compounds of formula (Ic), characterized in that:

the A-D chain formation comprises a Ze group and a Wiβ group, whilst the B-C chain formation comprises a Ze group and a Wiα group,

or the A-D chain formation comprises a Ziβ group and a We group, whilst the B-C chain formation comprises a Ze group and a Wiα group,

or the A-D and B-C chain formations each comprise a Ziβ group and a We group.

Preferred compounds of formula (Ic) are those characterized by the following formulae:

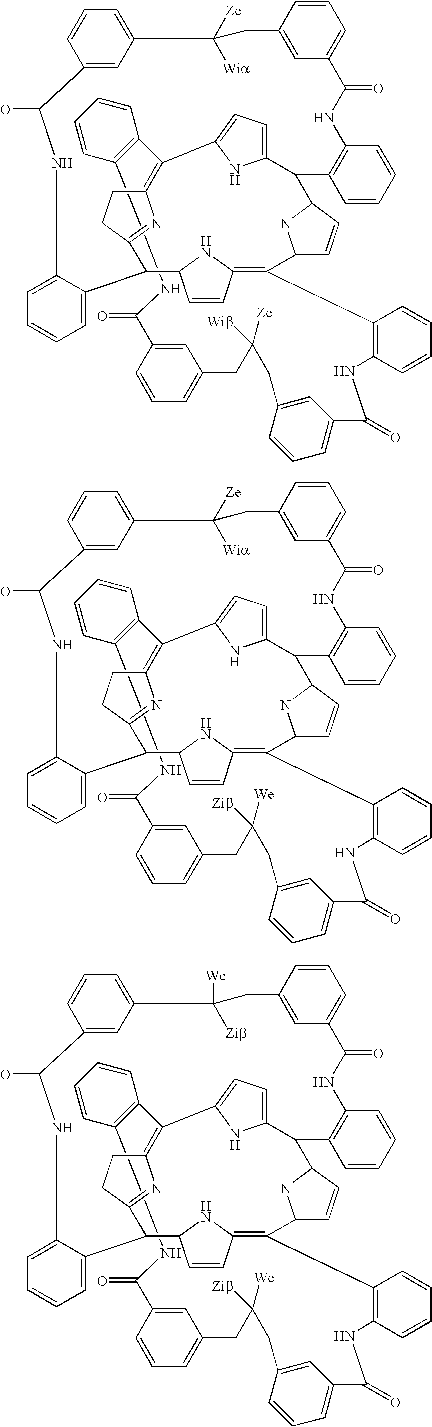

The invention also relates to the compounds of formula (I), and more particularly those of formula (Ia), (Ib), and (Ic), as defined above, in which Z represents a CH₂NR₁R₂ group, in which at least one of R₁ and R₂ represent a specific antibody, if appropriate linked to the CH₂N part of said group via a spacer.

Such antibodies can be chosen from those mentioned at the Roche Symposium, held on Thursday 7th Jun. 2001, Paris, EUROCANCER 2001, in particular from the following antibodies:

J591: murine IgG2A, anti-PSMA (Prostate Specific Membrane Antigen) expressed on carcinomatous human prostate cells,

B4: murine IgG1, anti-CD19 expressed on Ramos and Daudi lymphoma cells,

HuM195: humanized IgG1, anti-CD33 expressed on human leukemia HL60 cells,

3F8: murine IgG3, anti-CD2 expressed on human neuroblastoma NMB7 cells,

Herceptin, trastuzumab: humanized IgG1, anti-HER2 expressed by human MCF7 breast and SKOV3 ovarian carcinoma cells,

35A7: directed against the carcinoembryonic antigen (CEA),

Basiliximab, Simulect: entirely chimeric, anti-CD25, used in the prevention of kidney graft rejection,

Gentuzumab ozogamicin: entirely humanized, anti-CD33,

Rituximab, Tositumomab: chimeric, anti-CD20, antigen expressed in more than 95% of neoplasic lymphocytes,

BL22: anti-CD22,

SGN-10: anti-LeY, expressed by different types of carcinoma, in particular by digestive epithelial cells and by pancreatic acinus cells.

By way of illustration, the abovementioned spacer is chosen from the groups of the following formulae:

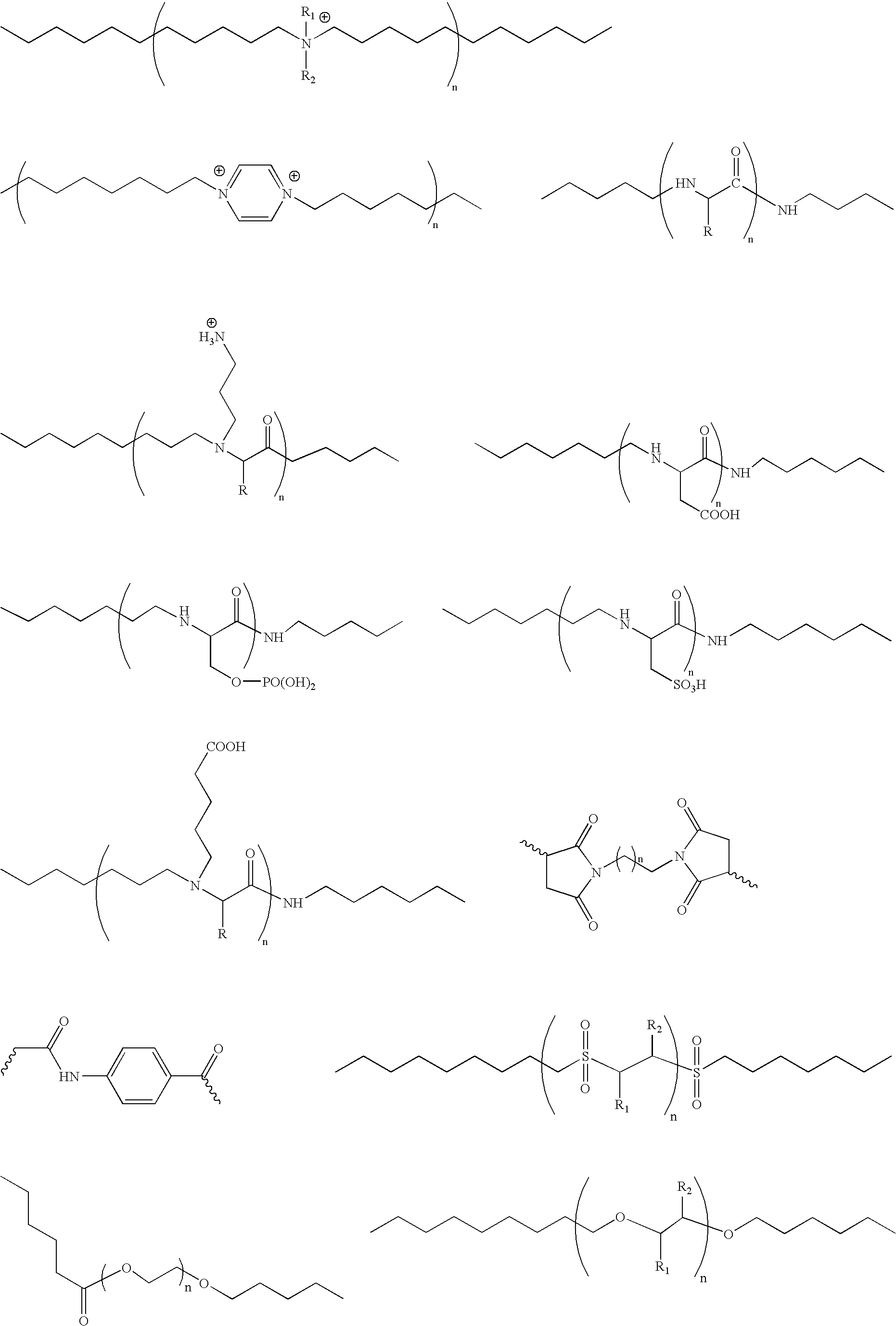

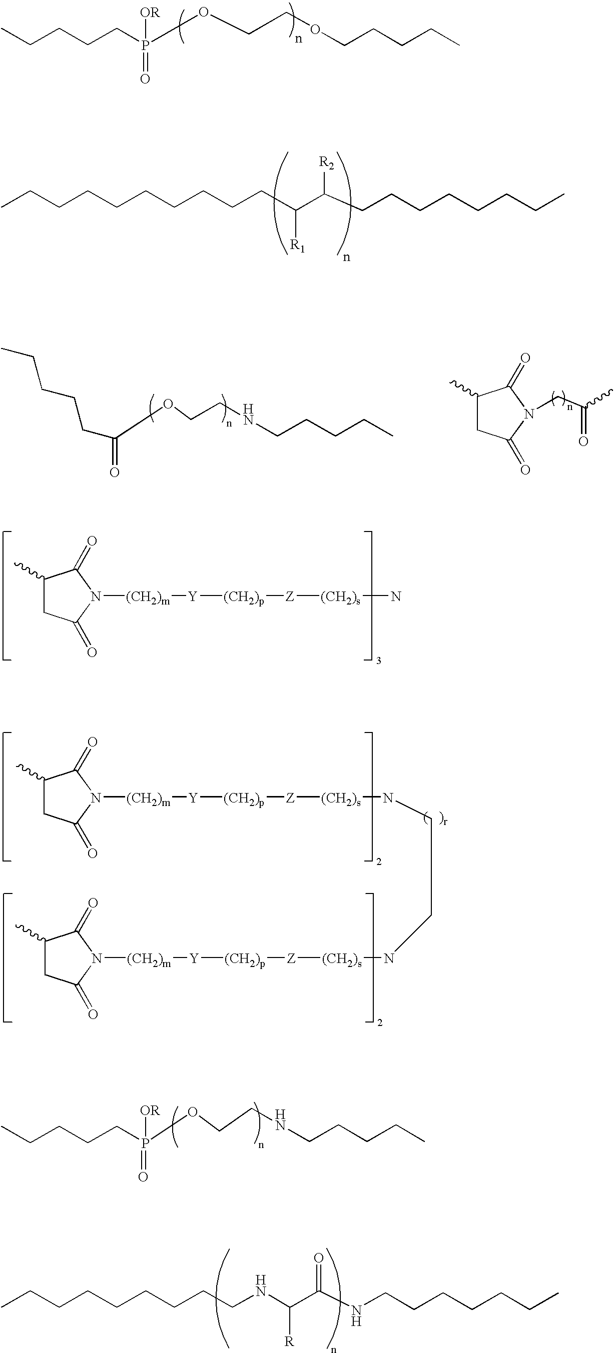

in which n and r represent an integer varying from 1 to 10, R₁ and R₂ represent, independently of one another, H, or a linear, branched, or cyclic alkyl group, with 1 to 8 carbon atoms, or an aryl or alkylaryl group, R represents one of the side chains of the 20 natural amino acids, Y and Z represent heteroatoms such as O or S, m, p and s, independently of each other, represent 0 or an integer varying from 1 to 10.

The invention also relates to complexes between a compound as defined above, and a radioelement chosen from the α emitters, or a divalent or trivalent metallic element.

A more particular subject is the abovementioned complexes between a compound as defined above, and an α-emitter radioelement chosen from bismuth-212 or -213, actinium-225, or astatine-211.

The invention also more particularly relates to the abovementioned complexes between a compound as defined above, and a divalent or trivalent metallic element chosen from Y(III), In(III), Cd(II), Mg(II), Mn(III), Fe(III), B(III) and the lanthanides.

In the complexes of the invention, the metals are situated in the centre of the porphyrin nucleus of the abovementioned compounds, but not necessarily in the porphyrin plane, and are bound to the nitrogen atoms of said nucleus by covalent bonds, two of which are of dative type.

A subject of the invention is also any pharmaceutical composition characterized in that it comprises a complex as defined above, in combination with a pharmaceutically acceptable vehicle.

Advantageously the pharmaceutical compositions according to the invention are presented in a form which can be administered by intravenous route.

Preferably, the abovementioned pharmaceutical compositions are characterized in that the dosage is approximately 15 to 50 mCi per patient divided into 3 to 6 fractions over 2 to 4 days.

The invention also relates to the use of complexes as defined above for the preparation of a medicament intended for the treatment of cancers, or for the preparation of compositions intended for medical imaging.

A more particular subject of the invention is the use of complexes as defined above, for the preparation of a medicament intended for the treatment of tumorous small-cell cancers, such as acute myeloid leukemia, non-Hodgkin's lymphomas, bronchopulmonary dysplasias, metastatic breast cancers, colorectal cancers, lymphomas, and pathologies in which the following antigenic units: CD52, CD22, CD20, HLA-DR, CD33, LE-Y, Ep-CAM, ACE, CAN, EGFR, KSA, VEGF, HER2, GD2, tenascin are involved.

The invention also relates to a process for preparing the abovementioned compounds of formula (I), characterized in that it comprises the following stages:

treatment of the compound of the following formula (II)

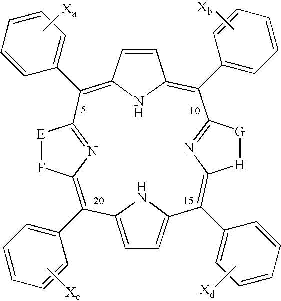

in which Xₐ, X_{b}, X_{c}, and X_{d}, represent NH₂, OH, COOH or CH₂Cl, and E, F, G, and H being defined above, said compound being such that:

Xₐ, X_{b}, X_{c}, and X_{d}, are in a position in the case of the synthesis of compounds of formula (Ia),

Xₐ, and X_{c}, are in α position, and X_{b} and X_{d} are in β position in the case of the synthesis of compounds of formula (Ib),

Xa, and Xd, are in β position, and X_{b} and X_{c} are in a position in the case of the synthesis of compounds of formula (Ic),

with a compound of formula Yₐ—C₆H₄—CH₂Cl in which Yₐ represents COOH, CH₂Cl, NH₂, or OH respectively,

a stage of treatment of the compound obtained at the preceding stage with a compound of formula Z—CH₂—W in which Z and W are as defined above, which leads to the obtaining of a compound of formula (I) the different variants of formulae (Ia), (Ib), and (Ic) of which are separated by purification, in particular by low pressure chromatography on silica gel, or preparative HPLC.

The above-mentioned complexes are obtained by bringing together the compounds of formula (I) with a radioelement as defined above.

The description is further illustrated by the detailed description which follows of particular compounds of the invention, and of the process for obtaining them.

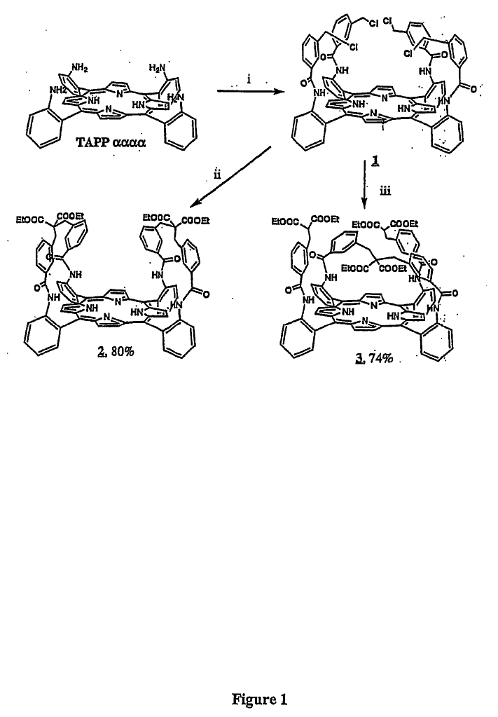

With regard to bismuth, given the properties of this metal, namely its azophilic and oxophilic character and high coordination number (up to nine atoms), the Inventors have synthesized models corresponding as well as possible to the requirements of the metal. In fact, the nitrogen atoms originating from the porphyrin ring are involved in the complexation of the metal and the handles bring groups possessing oxygen atoms to the metal. Moreover, it should be noted that the ligand forms a cage capable of accepting and stabilizing the metal. FIG. 1 represents the basic skeleton of ligands, as well as the process for synthesis of the latter.

The provision of this type of porphyrin compared with picket porphyrins (Buckingham, D.; Clarck, C.; Webley, W. J Chem. Soc. Chem. Com. 1981, 192, Michaudet, L.; Richard, P.; Boitrel, B. Chem. Commun. 2000, 1589-1590, Michaudet, L. doctoral thesis, University of Burgundy, Jul. 12, 2000, Dijon) resides in the pre-organization of the handle (or handles). The fact that the pickets or the handles are pre-organized makes it possible to have a carboxylic-type group (acid, or ester) just above the metal. Moreover, thanks to the modularity of the synthesis, it is possible to envisage varying the number of coordinating groups.

This diagram, although representing the basis for the present invention, is not applicable and has already been published (Didier, A.; Michaudet, L.; Ricard, D.; Baveux Chambenoit, V,; Richard, P.; Boitrel, B. Eur. J Org. Chem. 2001, 1917-1926), for two different reasons. On the one hand, after saponification of the ester functions (and consequently decarboxylation), there is no control of the position of the residual acid function. On the other hand, these porphyrins possess no subsequent functionalization point, necessary for grafting to an antibody, or for making the compound hydrosoluble.

On the other hand, this type of ligand possesses a very rigid structure, with a predetermined geometry, which should increase the stability of the complex formed. It is in fact for reasons of stabilization of the metallic element that the synthesis of the cyclohexylbenzyl DTPA was developed, this ligand being more rigid in nature than DTPA (Brechbiel, M.; W.; Gansow, O. A. J. Chem. Soc., Perkin Trans. I 1992, 1173).

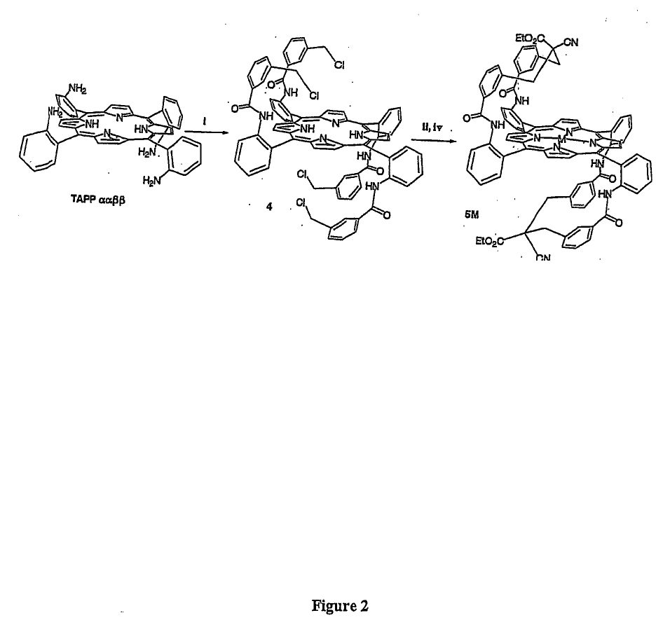

In order to avoid obtaining two different products as represented in FIG. 1, the same synthesis strategy was applied to the ααββ isomer (FIG. 2).

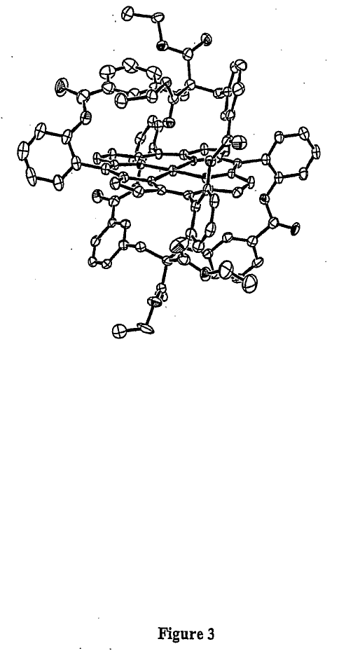

The obtaining of a radio-crystallographic structure clearly shows that the ethoxycarbonyl group directed towards the interior of the porphyrin is suitably maintained above the metal (FIG. 3).

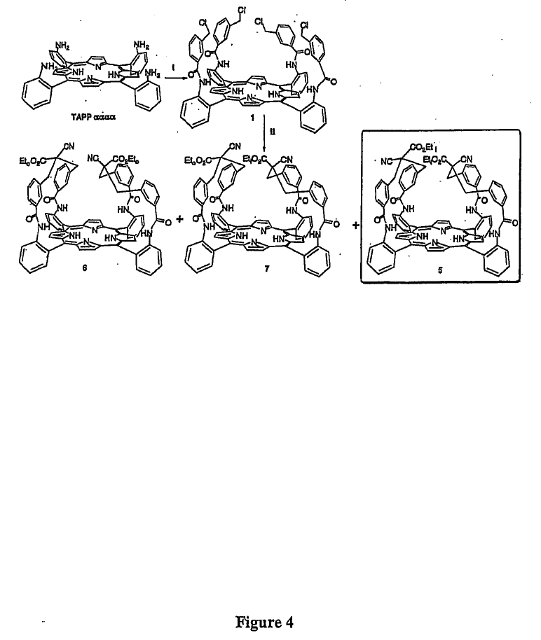

The invention consists of using ethyl cyanoacetate (NC—CH₂—CO₂Et) instead of ethyl malonate during the synthesis described in FIG. 1, and applying it to the ααββ and αβαβ isomers in addition to the αααα isomer. In the case of the αααα isomer, three porphyrins are thus obtained, from which porphyrin 5 can be purified, which possesses the ester function oriented towards the interior (FIG. 4).

This orientation of the ester function makes it possible to make these compounds useable for a coordination of metals such as bismuth (III) or the lanthanides. By proton spectroscopy, the structure 5 can be immediately attributed to a molecule due to its symmetry and the significant shielding undergone by the ethyl groups oriented towards the interior of the cavity. In fact, by analogy with the NMR spectroscopy spectrum of compound 2, compounds 5 and 6 can be easily discerned. Moreover, compound 7 represents a ligand of the same conformation, but with a single ethoxycarbonyl group for coordinating the metal.

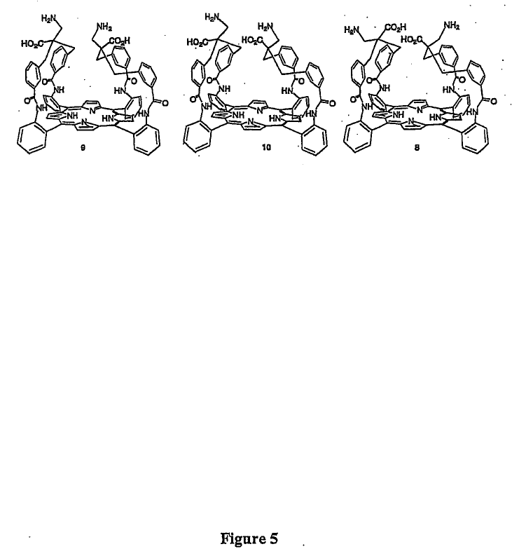

Therefore, after saponification in a first phase, and reduction of the CN function to CH₂—NH₂ in a second phase, products are obtained the conformation of which is perfectly known and which possess two subsequent functionalization points (FIG. 5).

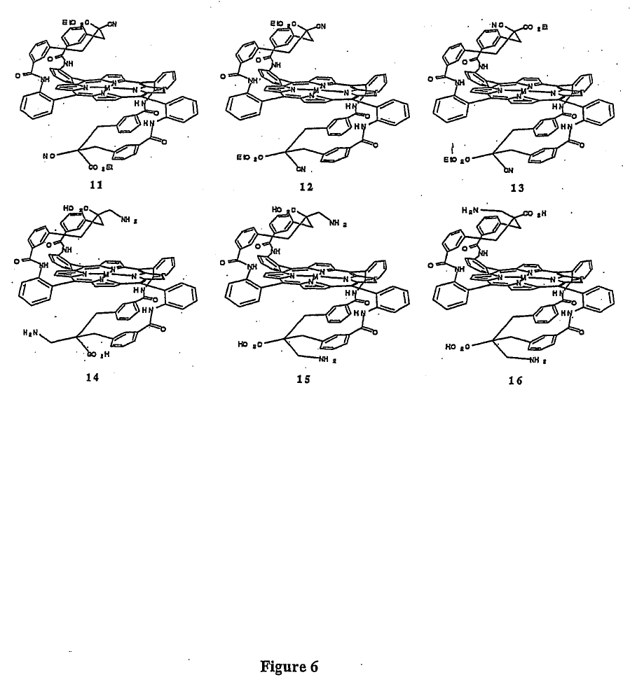

As described for FIG. 2, this variant applied to the ααββ atropisomer will give rise to the six ligands represented in FIG. 6.

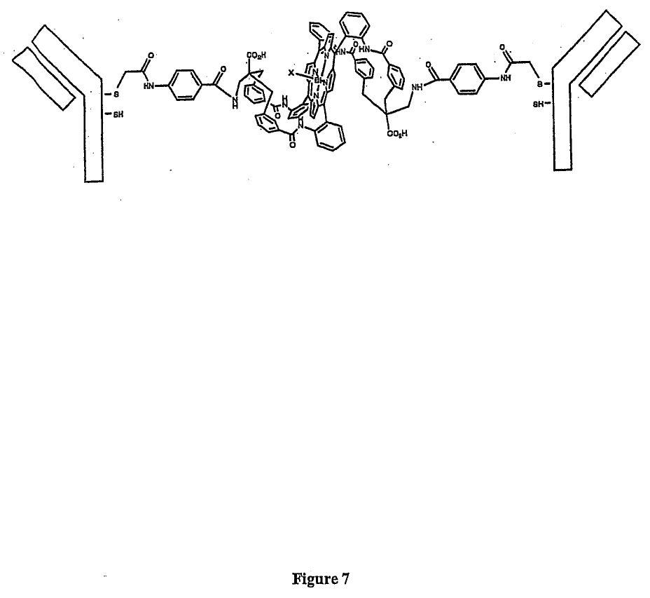

The usefulness of the porphyrins, represented in FIG. 6, resides in the identity of their two faces both for the coordination of the metal and for grafting to an antibody. This structure can be used for the construction of bispecific monoclonal antibodies. The latter result from the assembly of two “semi-antibodies” on a bifunctional spacer (such as a bismaleimide-type derivative). The porphyrin 16 can be considered in this context both as a complexing element and as a bridging element as represented diagrammatically in FIG. 7.

It should be noted that the porphyrin has been grafted onto two Fab′ fragments via a bifunctional spacer called SIAB for (N-succinimidyl(4-iodoacetyl)-aminobenzoate. These two fragments can be of different specificity in order to improve the recognition specificity.

It should also be noted that two thiol functions are presented on the Fab′ fragment, and that as a result different connection diagrams are possible between the porphyrin and the antibody.

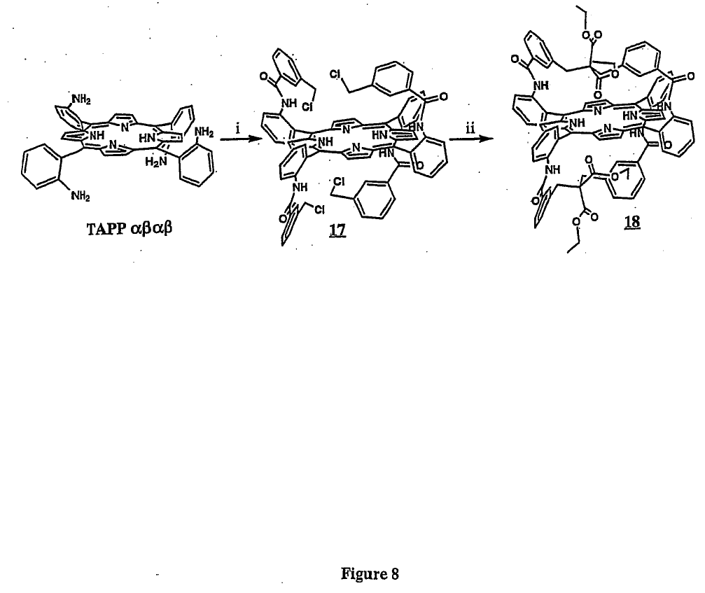

Finally, the fact of obtaining compound 3 (FIG. 1) from the αααα atropisomer demonstrates that the same reaction sequence applied to the αβαβ atropisomer gives rise to compound 18, of bis-ansa type (FIG. 8). This type of compound is useful for prohibiting any intermolecular interaction such as the formation of dimers as described for aliphatic picket porphyrins (Michaudet et al., 2000, mentioned above).

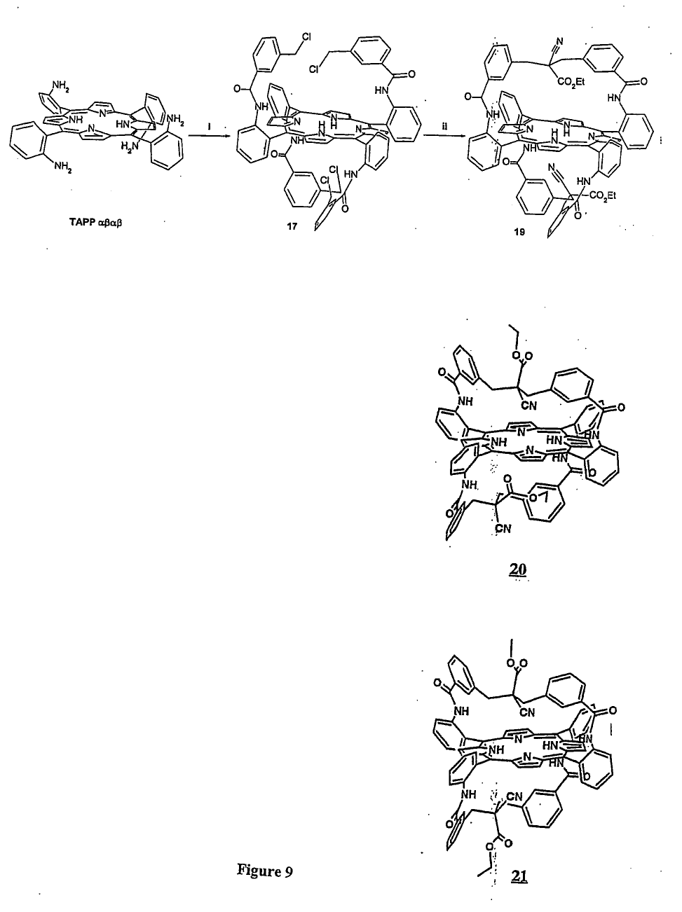

Molecular dynamic modelling of porphyrin 18 shows that the pre-organization of this superstructure perfectly directs one of the two carbonyl functions (belonging to the ester) towards the coordination centre. This point signifies that it is again possible to differentiate the “internal” from the “external” ethoxycarbonyl group, and therefore that if Stage ii) is carried out with ethyl cyanoacetate, three porphyrins 19, 20 and 21 (FIG. 9) are again obtained.

Experimental Part

α-5,10,15,20-Tetrakis{2-[(3-chloromethyl)benzoylamido]phenyl} porphyrin: 1

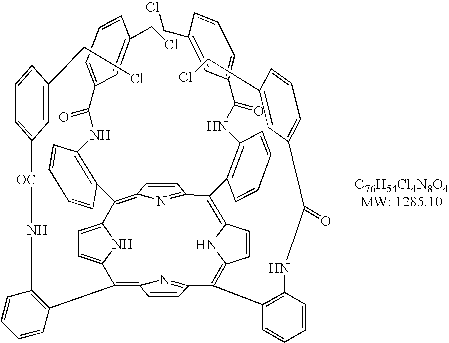

0.2 g of TAPP 4.0 (0.29 mmol), 0.5 mL of triethylamine and 20 mL of THF, are introduced into a 100-mL two-necked flask under argon. 0.34 mL (2.3 mmol) of 3-(chloromethyl) benzoic acid chloride are added using a syringe. The reaction is carried out at ambient temperature over 12 hours, then the reaction mixture is evaporated, followed by purification by chromatography on a silica column and the desired product is eluted with a mixture of methanol in dichloromethane (0.1%) then isolated with a yield of 81% (0.3 g, 0.23 mmol).

Elemental analysis: C₇₆H₅₄N₈Cl₄O₄, calculated (%): C, 71.03; H, 4.24; N, 8.72; found (%): C, 70.89; H, 4.11; N, 8.83

Mass spectrometry (FAB): m/z=1284.9 [M]^{+.}

Infra-red (KBr, cm⁻¹): 1680 (C=O)_{amide'}3415 (NH)

Mass spectrometry HRMS: calculated m/z=1304.2920 for C₅₆H₄₆N₈O8Na measured m/z=1304.2909

NMR ¹H (δ ppm, CDCI₃, 300 K: 8.99 (s, 8H, β-pyr.); 8.89 (d, J=8.3 Hz, 4H, aro.); 8.02 (dd, J=1.3 Hz, J=7.5 Hz, 4H, aro.); 7.92 (td, J=1.3 Hz, J=8.3 Hz, 4H, aro.); 7.81 (s, 4H, —NHCO); 7.59 (td, J=0.9 Hz, J=7.6 Hz, 4H, aro.); 6.52 (broad s, 4H, aro_{pick.}); 6.51 (d, J=8.1 Hz, 4H, aro_{pick}); 6.40 (d, J=7.7 Hz, 4H, aro_{pick}); 6.40 (d, J=7.7 Hz, 4H, aro_{pick}); 6.00 (t, J=7.7 Hz, 4H, aro_{pick}); 3.23 (s, 8H, —CH₂—); −2.47 (s, 2H).

NMR ¹³C (δ ppm, CDCI₃, 300 K): 165.2; 138.8; 137.5; 135.6; 135.2; 132.4; 131.3; 131.0; 130.6; 128.4; 126.6; 126.0; 123.9; 121.3; 115.5; 44.6.

α-5,10: α-15,20-Bis-{2,2′-[3,3′-(2,2-(diethoxycarbonyl)propane-1 ,3-diyl) dibenzoylamido]diphenyl}porphyrin: 2

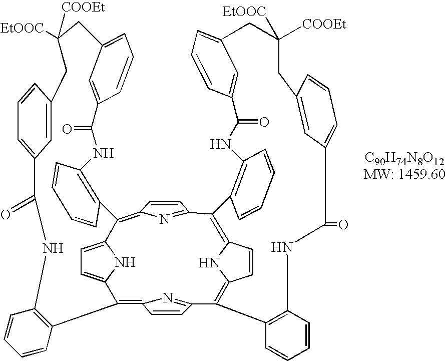

18 mg (0.8 mmol) of sodium is dissolved in 5 mL of absolute ethanol in a 50-mL three-necked flask under argon. After V₂ hour ethyl malonate (118 μl, 0.8 mmol) is added using a syringe. 50 mg of 1 (0.04 mml), are previously dissolved in 10 mL of THF, then added dropwise to the reaction mixture. The crude product is evaporated at the end of 24 hours, then deposited on a silica column. The product is eluted with a mixture of MeOH/CH₂Cl₂ and obtained with a yield of 80% (46 mg, 0.03 mmol).

Elemental analysis: C₉₀H₇₄N₈O₁₂•CH₂Cl₂, calculated (%): C, 70, 76; H, 4.96; N, 7.25 found (%): C, 70.68; H, 4.06; N, 6.93

Infra-red (KBr, cm⁻¹): 1726 (C=O)ₑₛₜₑᵣ, 1683 (C=O)_{amide}, 3417 (NH)

Mass spectrometry (FAB): m/z=1459.1 [M]^{+.}

Mass spectrometry HRMS: calculated m/z=1481.5335 for C₉₀H₇₄N₈NaO₁₂; measured m/z=1481.5324

NMR ¹H (δ ppm, CDCI₃, 300 K): 8.92 (s, 4H, β-pyr); 8.87 (s, 4H, , β-pyr.); 8.69 (d, J=8.4 Hz, 4H, aro.); 7.91 (td, J=7.8 Hz, J=1.3 Hz, 4H, aro.); 7.79 (dd, J=7.5 Hz, J=1.2 Hz, 4H, aro.); 7.49 (td, J=7.5, 4H, aro.); 7.41 (s,4H, —NHCO); 7.07 (d, J=7.9 Hz 4H, aro_{pick}); 6.65 (d, J=7.5 Hz, 4H, aro_{pick.}); 6.59 (s, 4H, aro_{pick.}); 6.39 (t, J=7.7 Hz, 4H, aro_{pick.}); 3.98 (q, J=6.9 Hz, 4H, —CH₂CH₃); 3.53 (q, J=7.1 Hz, 4H, —CH₂CH₃); 2.38 (d, J=13.8, 4H, —CH₂—); 2.19 (d, J=13.8 Hz, 4H, —CH₂—); 1.09 (t, J=6.9Hz, 6H, —CH₂CH₃); 0.64 (t, J=7.1 Hz, 6H, —CH₂CH₃); −2.95 (s, 2H).

NMR ¹³C (δ ppm, CDCI₃, 300 K): 170.9; 170.5; 166.7; 138.3; 136.5; 135.9; 134.7; 133.2; 132.1; 130.3; 128.6; 127.8; 126.3; 123.7; 122.7; 115.4; 31.7; 61.7; 61.4; 40.7; 14.4; 13.8.

α-5,15-{2,2′-[3,3′-(2,2-(diethoxycarbonyl)propane-1,3-diyl)dibenzoylamido]diphenyl}: α-10,20-Bis-{2,2 ′-[3,3′-(1,1-(diethoxycarbonyl)ethane-2yl) benzoylamido]phenyl} porphyrin: 3

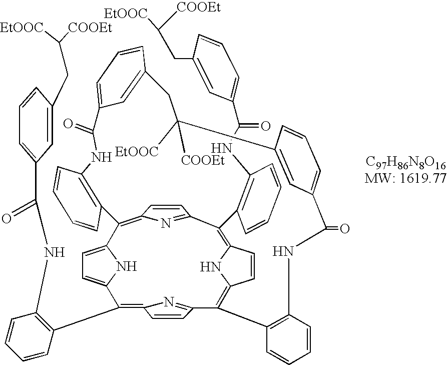

The same operating method as that adopted in order to synthesize the preceding molecule is implemented. Starting with 0.89 of sodium (40 mmol) and 5.9 mL of ethyl malonate (40 mmol) in 35 mL of absolute ethanol, 50 mg (0.04 mmol) of porphyrin 1 dissolved in 10 mL of THF is added. The crude product is chromatographed on a silica column and the desired product is eluted with a mixture of pentane/chloroform (5/100) with a yield of 74% (47 mg, 0.03 mmol).

Elemental analysis: C₉₇H₈₆N₈O₁₆•H₂O, calculated (%): C,71.14; H,4.42; N, 6.84; found (%): C, 69.92; H, 4.25; N, 6.63

Mass spectrometry (MALDITOF): m/z=1619.3 [M]^{+.}

Infra-red (KBr, cm⁻¹): 1732 (C=O)_{ester'}1682 (C=O)_{amide'}3416 (NH)

NMR¹H (δ ppm, CDCI₃, 320 K): 9.08 (d, J=8.44 Hz, 2H, aro); 9.06 (d, J=4.7 Hz, 4H, β-pyr.); 8.95 (d, J=4.7 Hz, 4H, β-pyr); 8.71 (d, J=8.4 Hz, aro.); 8.56 (broad s, 2H, —NHCO); 8.04 (dd, J=1.1 Hz, J=6.9 Hz, 2H, aro.); 7.94 (broad t, J=6.6 Hz, 4 H, aro., —NHCO); 7.85 (td, J=1.3 Hz, J=8.2 Hz, 2H, aro.); 7.69 (d, J=7.7 Hz, 2H, aro_{pick.}); 7.65 (dd, J=1.3 Hz, J=7.7 Hz, 2H, aro.); 7.59 (t, J=7.2 Hz, 2H, aro.); 7.55 (s, 2H, aro_{pick.}); 7.47 (t, J=7.5 Hz, 2H, aro.); 6.97 (t, J=7.7 Hz, 2H, aro_{pick.}); 6.50 (d, J=7.8 Hz, 4H, aro_{pick.}); 6.44 (d, J=5.7 Hz, 2H, aro_{pick.}); 5.88 (t, J=7.4 Hz, 2H, aro_{pick.}); 4.84 (s, 2H, aro_{pick.}); 4.09 (m, 8H, —CH₂—CH₃); 3.38 (t, J=7.6 Hz, 2H, —CH₂CH—); 2.81 (d, J=7.6 HZ, 4H, —CH₂CH—); 1.63 (s, 4H, —CH₂—); 1.17 (t, J=7.2 Hz, 12H, —CH₂CH₃); 0.95 (broad s, 4H, —CH₂, CH₃); −0.6 (broad s, 6H, —CH₂CH₃); −2.25 (s, 2H).

NMR ¹³C (δ ppm, CDCI₃, 300 K): 168.8; 168.1; 166.6; 164.6; 139.2; 138.9; 138.0; 137.7; 136.1; 135.4; 135.2; 133.7; 132.9; 132.5; 131.9; 131.4; 130.6; 130.3; 128.8; 128.7; 128.5; 127.7; 125.8; 123.9; 123.5; 122.6; 120.9; 116.5; 115.1; 62.0; 53.6; 42.0; 34.1; 14.2; 12.0.

α-5,10: β-15,20-Tetrakis{2-[(3-chloromethyl)benzoylamido]phenyl}porphyrin: 4

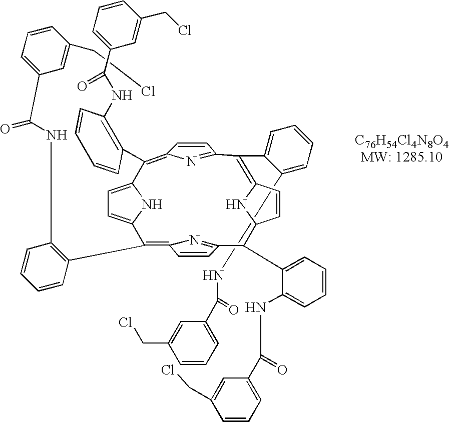

0.674 g (1 mmol) of ααββ TAPP, 2.22 mL (16 mmol) of triethylamine and 100 mL of THF are introduced into a 250-mL two-necked flask, under argon. 0.71 mL (5 mmol) of 3-(chloromethyl)benzoic acid chloride dissolved in 10 mL of THF are added dropwise. The reaction is carried out at 0° C. for 3 hours, then the reaction mixture is evaporated. The residue is purified by chromatography on a silica column, the product is eluted with pure dichloromethane, then isolated with a yield of 86% (1.10 g).

NMR ¹H 500 MHz (δ ppm, CDCI₃, 300 K): −2.52 (s, 2H, NH_{pyr}); 3.52 (d, 4H, Jo=12.1 Hz, (CH₂)_{benz}); 3.55 (d, 4H, Jo=12.1 Hz (CH₂)_{benz}); 6.39 (t, 4H, Jo=7.7 Hz, aro_{pick}); 6.52 (d, 4H, Jo=8.3 Hz, aro_{pick}); 6.55 (s, 4H, aro_{pick}); 6.74 (d, 4H, J₀=7.5 Hz, aro_{pick}); 7.61 (t, 4H, Jo=7.5 Hz, aro); 7.66 (s,4H, NHCO); 7.93 (t,4H, Jo=8.3 Hz, aro); 8.07 (d, 4H, Jo=7.3 Hz, aro); 8.90 (d, 4H, Jo=8.3 Hz, aro); 8.99 (s, 4H, βpyr); 9.00 (s, 4H, βpyr).

NMR ¹³C 125 MHz (δ ppm, CDCI₃, 300 K): 44.8; 115.4; 121.5; 123.9; 126.3; 126.6; 128.7; 130.7; 131.3; 135.2; 135.5; 137.6; 138.8; 165.2.

UV-vis (CH₂Cl₂, λ/nm (10⁻³.ε, M⁻¹.cm⁻¹)): 422 (363.8); 515 (20.6); 549 (5.1); 589 (6.2); 646 (2.6).

Mass spectrometry (SMHR, LSIMS) calculated m/z=1305.2920 [M+Na]⁺ for C₇₆H₅₄Cl₄N₈NaO₄, found 1305.2899.

Elemental analysis: for C₇₆H₅₄Cl₄N8O₄, calculated (%): C, 71.03; H, 4.24; N, 8.72; found (%): C, 70.62; H, 4.19; N, 8.94.

Infrared (KBr, υ cm⁻¹): 3420 5NH); 1684 (CO).

α-5,10:β-15,20-Bis{2.2′-[3.3′-(2.2′-(diethoxycarbonyl)propane-1,3-diyl) dibenzoylamido]diphenyl}porphyrin: 5

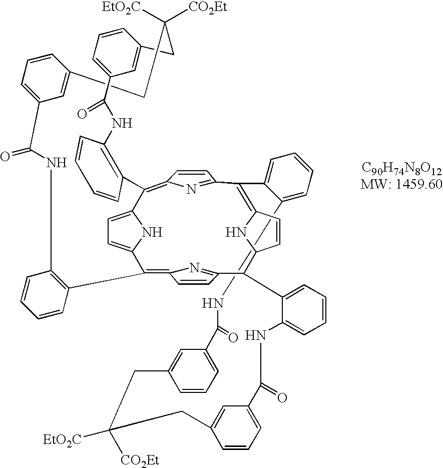

0.19 mg (8.2 mmol) of sodium is dissolved in 30 mL of absolute ethanol in a 50-mL three-necked flask under argon. After 30 minutes, ethyl malonate (1.24 mL, 8.2 mmol) is added using a syringe. 0.35 g (0.27 mmol) of 4 are previously dissolved in 20 mL of THF, then added dropwise to the reaction mixture. The crude product is evaporated at the end of 2 hours, then the residue is deposited on a silica column. The product is eluted with dichloromethane and obtained with a yield of 75% (0.30g).

NMR¹H 500 MHz (δ ppm, CDCI₃, 323 K ): −2.16 (s, 2H, NH_{pyr}), −0.03 (t, 6H, J=7.0 Hz, CH₂(CH₃)ᵢ); 0.57 (d, 4H, Jo=13.7 Hz, (CH₂)_{benz}), 0.63 (t, 6H, Jo=7.0 Hz, CH₂(CH₃)₀); 1.44 (d, 4H, Jo=13.5 Hz, (CH2)_{benz}); 2.46 (q, 4H, Jo=7.0 Hz, (CH₂)ᵢCH₃); 3.29 (q, 4H, Jo=7.0 Hz, (CH₂)C1CH₃); 4.84 (s, 4H, aro_{pick}); 6.61 (d, 4H, Jo=7.6 Hz, aro_{pick}); 6.93 (t, 4H, Jo=7.6 Hz, aro_{pick}); 7.42 (s, 4H, NHCO); 7.51 (td, 4H, Jo=7.6 Hz, Jm=1.2 Hz, aro_{pick}); 7.55 (td, 4H, Jo=7.3 Hz, Jm=1.6 Hz, aro); 7.87 (td, 4H, Jo=7.3 Hz, Jm=1.0 Hz, aro); 7.89 (dd, 4H, Jo=8.4 Hz, aro); 8.69 (s, 4H, β-pyr); 8.70 (dd, 4H, Jo=8.4 Hz, Jm=1.0 Hz, aro); 8.98 (s, 4H, β-pyr).

NMR ¹³C 125 MHz (δ ppm, CDCI₃, 323 K): 12.9; 13.7; 40.5; 59.5; 60.3; 60.9; 115.2; 122.8; 123.9; 126.1; 127.2; 128.6; 130.3; 130.6; 132.4; 133.0; 133.4; 134.0; 134.9; 135.9; 139.0; 165.1; 168.9; 169.7.

UV-vis (CH₂Cl₂, λ/nm (10⁻³.ε M⁻¹.cm ⁻¹)): 422 (433.5); 516 (17.4); 550 (4.8); 590 (5.6); 647 (1.4).

Mass spectrometry (FAB): m/z=1458.6 [M]⁺.

Elemental analysis: for C₉₀H₇₄N₈O₁₂, calculated (%): C, 74.06; H, 5.11 ; N, 7.68; found (%): C, 74.25; H, 5.35; N, 7.30.

Infrared: (KBr, ν cm⁻¹): 3426 (NH); 1728 (CO); 1686 (CO). 5Ni (radiocrystallographic structure of FIG. 3)

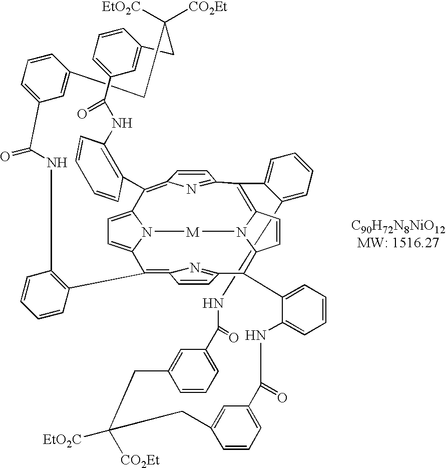

45 mg of 5 are dissolved in 1.5 mL of pyridine. An excess of nickel acetate is added. The solution is taken to reflux for 1 hour, then the solvents are evaporated off. The residue is dissolved in CH₂Cl₂, filtered and dried again. After chromatography on a silica column (eluent: CH₂Cl₂/MeOH (98/2), the desired product is obtained with a yield of 98%).

NMR ¹H MHz (δ ppm, CDCI3, 323 K): 0.16 (t, 6H, Jo=7.1 Hz, CH₂(CH₃)ᵢ); 0.70 (t, 6H, Jo=7, Hz, CH₂(CH₃)ₒ); 1.01 (d, 4H, Jo=13.7 Hz, (CH2)benz); 1.66 (d, 4H, Jo=13.7 Hz, (CH₂)_{benz}); 2.81 (q, 4H, J=7.6 Hz, aro_{pick}); 3.41 (q, 4H, J=7.1 Hz, (CH₂)oCH₃); 4.85 (s, 4H, aro_{pick}); 6.67 (d,4H, J =7.6 Hz, aro_{pick}); 6.96 (t, 4H, J=7.6 Hz, aro_{pick}); 7.34 (s, 4H, NHCO); 7.47 (td, 4H, Jo=7.6 Hz, Jm =0.9 Hz, aro_{pick}); 7.59 (td, 4H, Jo=7.7 Hz, Jm=1.4 Hz, aro); 7.77 (dd, 4H, Jo=7.6 Hz, Jm =1.2 Hz, aro); 7.82 (td, 4H, Jo=8.1 Hz, Jm=1.2 Hz, aro); 8.65 (s, 4H, β-pyr); 8.70 (dd, 4H Jo=8.1 Hz, Jm=0.9 Hz, aro); 8.85 (s, 4H, βpyr).

NMR 13C 125 MHz (δ ppm, CDCI₃, 323 K): 13.1; 13.8; 40.6; 60.6; 61.0;; 114.5 122.4; 123.8; 126.2; 127.3; 128.7; 130.3; 132.7; 133.0; 133.4; 133.9; 134.1; 136.0; 138.7; 164.7; 170.0.

Mass spectrometry (MALDI/TOF) m/z=1514.71 [M]⁺.

Elemental analysis: for C₉₀H₇₂N₈NiO₁₂•H₂O, calculated (%): C, 70.45; H, 4.86; N, 7.30; found (%): C, 70.54; H, 5.21; N, 7.06.

Infrared (KBr, ν cm⁻¹): 3419 (NH); 1687 (CO).

5Zn

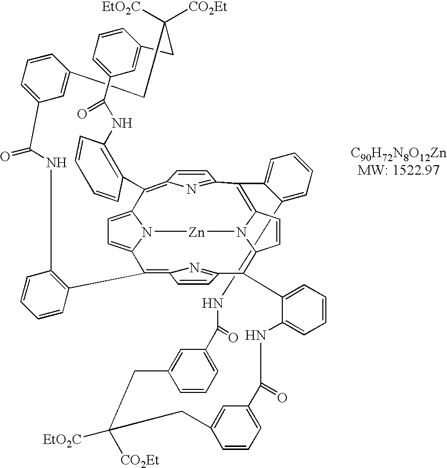

This complex was prepared from 5, according to the following process. 50 mg of porphyrin base are dissolved in 10 mL of a CHCl₃/MeOH mixture (2%). An excess of dihydrated zinc acetate and sodium acetate are added. The solution is taken to reflux for 1 hour, then the solvents are evaporated off. The residue is dissolved in CH₂Cl₂, filtered and dried again. After chromatography on a silica column (eluent: CH₂Cl₂/MeOH (97/3)), a pink-violet product is isolated with a quantitative yield (98%).

NMR ¹H 500 MHz (δ ppm, CDCI₃, 323 K): −0.40 (d, 4H, Jo=13.0 Hz, (CH₂)_{benz}); 0.11 (t, 6H Jo=7.1 Hz; CH₂(CH₃)ᵢ); 0.21 (t, 6H, Jo=7.1 Hz, CH₂(CH₃)ₒ); 1.31 (d, 4H, Jo=13.0 Hz, (CH₂)_{benz}); 2.27 (q, 4H, Jo=7.2 Hz, (CH₂)ᵢCH₃); 2.46 (q, 4H, Jo=7.2 Hz, (CH₂)ₒCH₃); 3.82 (s, 4H, aro_{pick}); 6.54 (td, 4H, Jo=7.7 Hz, Jo=1.3 Hz aro_{pick}); 6.94(t, 4H, Jo=7.7 Hz, aro_{pick}); 7.26 (s, 4H, NHCO); 7.55 (td, 4H, Jo=7.6 Hz, Jm=1.2 Hz, aro_{pick}); 7.62 (td, 4H, Jo=8.1 Hz, Jm=1.6 Hz, aro); 7.86 (td, 4H, Jo=8.1 Hz, Jm=1.5 Hz, aro); 7.99 (dd, 4H, Jo=7.5 Hz, Jm=1.2 Hz, aro); 8.69 (dd, 4H, Jo=8.3 Hz, Jm=0.9 Hz, aro); 8.79 (s, 4H, β-pyr); 9.03 (s, 4H, βpyr).

NMR ¹³C 125 MHz (δ ppm, CDCI₃, 300 K): 13.1; 13.3; 40.5; 60.4; 115.8; 122.4 123.9; 124.7; 127.6; 128.8; 130.1; 132.7; 132.9; 133.0; 133.7; 133.9; 134.5; 139.2; 151.1; 151.9; 164.3; 168.2; 170.1.

Mass spectrometry (MALDI/TOF): m/z=1522.01 [M+H]⁺.

Elemental analysis: for C₉₀OH₇₂N₈O₁₂Zn•2H₂O, calculated (%): C, 69.34; H, 4.91; N, 7.19; found (%): C,69.09; H, 4.90; N, 7.43.

Infrared (KBr, ν cm⁻¹): 3420 (NH); 1728 (CO); 1683 (CO)

Preparation of Compounds 11, 12 and 13 (see FIG. 6)

Experimental Conditions

715 mg (31.1 mmol) of sodium is dissolved in 45 mL of absolute ethanol in a 250-mL flask under argon. After 1 hour, ethyl cyanoacetate (3.32 mL; 31.1 mmol) is added using a syringe. The solution whitens after a few minutes. The mixture is maintained under stirring for 1 hour. 400 mg (0.311 mmol) of 4 are previously dissolved in 70 mL of THF, then added dropwise to the reaction mixture. The crude product is evaporated after 12 hours, then the residue is precipitated from a mixture of dichloromethane and pentane. The precipitate is dissolved in a minimum amount of dichloromethane in order to be deposited on a silica column. A progressive rise to 0.2 % methanol makes it possible to obtain the expected 3 products according to the relative position of the CN and CO₂Et groups. Several chromatography columns are necessary in order to obtain these 3 products of satisfactory purity with the following yields: (8 mg, 2%), (70 mg, 17%) (150 mg, 36%).

The overall yield of the reaction is evaluated at 72 %.

Characterization

Product obtained at 2%:

NMR ¹H (500 MHz, CDCl₃, 300 K): δ=8.96 (s, 4H, β-pyr); 8.75 (d, J=8.2 Hz, 4H); 8.62 (s, 4H, β-pyr); 7.87 (m, 8H); 7.71 (d, J=7.9 Hz, 4H); 7.54 (t, J=7.7Hz, 4H); 7.31 (s, 4H, NH); 7.08 (t, J=7.7 Hz, 4H); 6.77 (d, J=7.1 Hz, 4H); 4.42 (s, 4H, H_{2′}); 1.90 (q, J=7.1 Hz, 4H, CH₂CH₃); 1.53 [d, J=12.9 Hz, 4H, CH₂]; −0.20 [br, 4H, CH₂]; −0.56 (t, J=7.1 Hz, 6H, CH₂CH₃); −1.96 (s, 2H).

ESI-HRMS: m/z calculated=1387.48063 [M+Na]⁺; found 1387.4809.

UV-VIS (CH₂Cl₂, λ nm, 10⁻³ ε, M⁻¹.cm⁻¹): 424 (221.0); 518 (14.2); 552 (3.5); 593 (3.8); 651 (0.9).

Product obtained at 17%:

NMR ¹H (500 MHz, CDCl₃, 300 K): δ=8.98 (s, 2H, β-pyr); 8.96 (s, 2H, β-pyr); 8.84 (d, J=8.2 Hz, 2H); 8.70 (d, J=4.7 Hz, 2H, β-pyr); 8.62 (d, J=4.7 Hz, 2H, β-pyr); 8.52 (d, J=8.2 Hz, 2H); 8.34 (d, J=7.4 Hz, 2H); 7.92 (t, J=7.9 Hz, 2H); 7.83 (t, J=7.9 Hz, 2H); 7.75 (d, J=7.9 Hz, 2H); 7.71 (t, J=7.9 Hz, 2H); 7.61 (d, J=7.7 Hz, 2H); 7.57 (s, 2H, NH); 7.54 (d, J=7.4 Hz, 2H); 7.40 (t, J=7.4 Hz, 2H); 7.08 (t, J=7.7 Hz, 2H); 6.94 (t, J=7.7 Hz, 2H); 6.89 (s, 2H, NH); 6.74 (d, J=7.7 Hz, 2H); 6.38 (d, J=7.7 Hz, 2H); 5.18 (s, 2H, H_{2′}); 3.58 (s, 2H, H_{2′}); 2.97 [q, J=7.1 Hz, 2H, (CH₂)ₑCH₃]; 2.10 [q, J=7.1 Hz, 2H, (CH₂)ᵢCH₃]; 1.77 [d, J=12.9 Hz, 2H, CH₂]; 1.39 [d, J=12.9 Hz, 2H, CH₂]; 0.83 [d, J=12.9 Hz, 2H, CH₂]; 0.11 [t, J=7.1 Hz, 3H, CH₂(CH₃)ₑ]; −0.56 [t, J=7.1 Hz, 3H, CH₂(CH3)i]; −1.91 [d, J=12.9 Hz, 2H, CH₂]; −2.04 (s, 2H).

ESI-HRMS: m/z calculated =1387.48063 [M+Na]⁺; found 1387.4804.

UV-VIS (CH₂Cl₂, λ nm, 10⁻³ ε, M⁻¹.cm⁻¹): 424 (308.5); 518 (17.1); 552 (4.0); 592 (4.9); 648 (1.3).

FTIR (KBr, cm⁻¹): 2240 (ν_{CN}).

Product obtained at 36%:

NMR ¹H (500 MHz, CDCI₃, 300 K): δ=9.06 (s, 4H, β-pyr); 8.71 (s, 4H, β-pyr); 8.54 (d, J=8.2 Hz, 4H); 8.10 (d, J=7.4 Hz, 4H); 7.89 (t, J=7.9 Hz, 4H); 7.62 (m, 8H); 7.08 (s, 4H, NH); 6.94 (t, J=7.4 Hz, 4H); 6.40 (d, J=7.7 Hz, 4H); 3.87 (s, 4H, H_{2′}); 2.94 (q, J=7.1 Hz, 4H, CH₂CH₃); 1.54 [d, J=12.4 Hz, 4H, CH₂]; 0.10 (t, J=7.1 Hz, 6H, CH₂CH₃); −1.41 [d, J=12.4 Hz, 4H, CH₂]; −1.96 (s, 2H).

$$\begin{matrix}ESI \text{-} HRMS \text{:} m \text{/} z = & calculated & {1387.48063 [ M + Na ]}^{+} \\ & found & 1387.4784\end{matrix}$$

NMR ¹³C (125 MHz, CDCl₃, 300 K): 165.6; 164.3; 138.7; 135.9; 134.2; 133.7; 132.8; 132.3; 131.4; 130.0; 129.1; 128.6; 128.0; 125.3; 124.2; 123.6; 115.3; 115.1; 61.6; 53.8; 40.2; 12.8.

UV-VIS (CH₂Cl₂, λ nm, 10⁻³ ε, M⁻¹.cm⁻³): 424 (353.2); 518 (18.3); 553 (4.0); 590 (5.3); 647 (1.6).

FTIR (KBr, cm⁻¹): 2240 (ν_{CN}).

Preparation of Compound 19 (see FIG. 9)

Experimental conditions:

215 mg (9.37 mmol) of sodium are dissolved in 12 mL of absolute ethanol in a 250-mL flask under argon. After stirring for I hour and 20 minutes, ethyl cyanoacetate (1.00 mL; 9.37 mmol) is added using a syringe. The solution whitens after a few minutes. The mixture is maintained under stirring for 1 hour. 120 mg (0.0937 mmol) of 17 (obtained from αβαβ TAPP, according to the protocol used for preparing 4 as described above) are previously dissolved in 30 mL of THF, then added dropwise to the reaction mixture over 30 minutes. The crude product is evaporated after 12 hours and washed with distilled water (3×50 mL). The aqueous phase is then precipitated from a mixture of dichloromethane and pentane. The precipitate is filtered and redissolved in a minimum amount of dichloromethane in order to be deposited on a silica column. A progressive rise to 0.2% methanol makes it possible to obtain product 19 with a yield of 60% (75 mg).

17: NMR ¹H (300 MHz, CDCl₃, 323 K): δ=8.95 (s, 8H, β-pyr); 8.84 (d, J=8.5 Hz, 4H, aro); 8.07 (d, J=8.5 Hz, J=1.2 Hz, 4H, aro); 7.92 (t, J=8.5 Hz, J=1.2 Hz, 4H, aro); 7.59 (t, J=8.5 Hz, 4H, aro); 7.46 (s, 4H, NH); 6.77 (d, J=7.5 Hz, 4H, aro); 6.65 (d, J=7.5 Hz, 4H, aro); 6.57 (t, J=7.5 Hz, 4H, aro); 6.30 (broad s, 4H, aro); 3.35 (s, 8H, CH₂); −2.51 (s, NH, 2H).

19: NMR ¹H (300 MHz, CDCl₃, 323 K): 3=9.14 (m, 2H, aro); 9.08 (s, 4H, β-pyr); 8.93 (s, 4H, β-pyr); 8.76 (s, 2H, aro); 8.07 (d, J=7.3 Hz, 2H, aro); 7.93 (m, 8H, aro); 7.79 (m, 6H, aro); 7.60 (t, J=7.4 Hz, 2H, aro); 7.52 (t, J=7.4 Hz, 2H, aro); 7.17 (m, 8H, aro+NH); 4.94 (s, 2H, H); 4.86 (s, 2H, H); 2.20 (m, 4H, (CH₂)); 1.62 (m, 4H, (CH₂)); −0.03 (m, br, 4H, CH₂CH₃); −1.46 (t, J=6.9 Hz, 6H, CH₂CH₃); −2.09 (s, 2H).

Metallation of Porphyrin by Bismuth in Ethanol

The usefulness of this process resides in the use of absolute ethanol. Moreover, the reaction takes place under air and at ambient temperature.

30 mg of free-base porphyrin (2.8×10⁻⁵ mol) is dissolved in 5 mL of ethanol. 30 mg of Bi(NO₃)₃, 5H₂O, (6.2×10⁻⁵ mol) is added. The solution instantaneously becomes coloured deep green. After stirring for 5 minutes, NH₃(g) is bubbled through for a few tens of seconds. The solution is evaporated and the mixture chromatographed on a silica column (eluent: 2% MeOH/dichloromethane). It will be noted that the residue is difficult to dissolve in dichloromethane. Once on the column, little free base to be separated is observed. The product is finally precipitated from a dichloromethane—pentane mixture. The yield is quantitative.

1. Compounds corresponding to the following general formula (I): 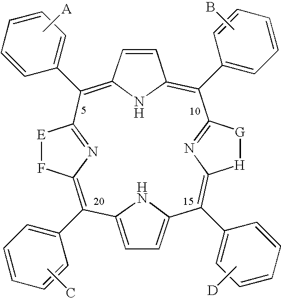
in which:
when A forms a chain with C, the so-called A-C chain, of formula (1) below:
—X—Y—C₆H₄—(CH₂)ₙ₁—U—(CH₂)ₙ₂—C₆H₄—Y—X— (1)
in which:
when X represents NH, O, CO or CH₂, Y represents respectively CO, CH₂, NH, or O,
n₁ and n₂, independently of one another represents an integer comprised between 1 and 3,
U represents a group of the C (Z, W) or N (CHRₐ-COOR_{b}) form, in which
Z represents:
an electroattractive group such as CN, NO₂, or CO₂,
or a CH₂NR₁R₂ group, in which R₁ and R₂ represent, independently of one another, H, or a linear, branched, or cyclic alkyl group, with 1 to 8 carbon atoms, or an aryl or alkylaryl group, or a specific antibody, if appropriate linked to the CH₂N part of said group via a spacer,
or an aryl group substituted by an SO₃R₃, SO₂R₃, P-NO₂ or o-NO₂ function, in which R₃ represents H, or a cation chosen from the alkali metals such as Na⁺, or K⁺, or R₃ represents an NR₄R₅ group in which R₄ and R₅ represent, independently of one another, a linear, branched, or cyclic alkyl group, with 1 to 8 carbon atoms, or R₃ represents a para-nitro aryl group,
W represents a CO₂⁻or COOR₆ group in which R₆ represents H or a linear, branched, or cyclic alkyl group, with 1 to 8 carbon atoms, or an aryl group, or an alcohol depopulated of electrons such as a para-nitro phenol or ortho-para-nitro phenol group,
or Z and W form in combination with the carbon atom which carries them a ring designated Meldrum's acid with the following formula: 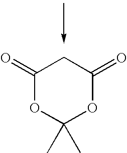
Rₐ corresponds to the definition previously given for R₁, or can also preferably represent the side chain of a natural or modified amino acid,
R_{b} corresponds to the definition previously given for R₁,
then B forms a chain with D, the so-called B-D chain, of the abovementioned formula (1), said A-C, and B-D chains, being situated independently of one another, above (α position) or below (β position) the porphyrin macrocycle plane,
or when A forms a chain with D, the so-called A-D chain, of the abovementioned formula (1), then B forms a chain with C, the so-called B-C chain, of the abovementioned formula (1), one of said A-D or B-C chains being situated above (ax position) the porphyrin macrocycle plane, whilst the other A-D or B-C chain, is situated below (I position) the porphyrin macrocycle plane,
E represents in combination with F, and H represents in combination with G, independently of each other, CH═CH, or CH₂—CH₂.

2. Compounds according to claim 1, characterized in that the chain formations of formula (1) are chosen from the following: 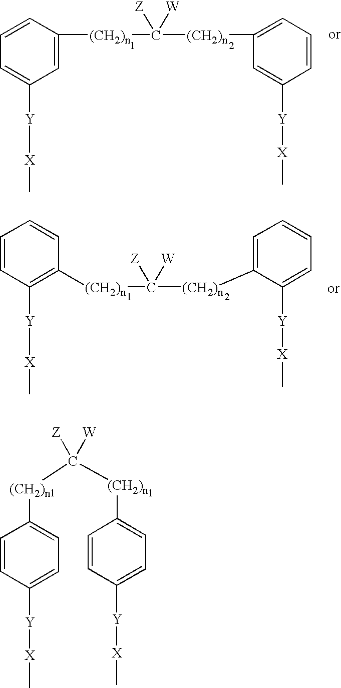
in which the Z and W groups are:
either directed towards the interior of said compounds and are situated above or below the porphyrin macrocycle plane according to whether said chain formations of formula (1) are situated respectively in α position or in β position, and are respectively designated Ziα and Wiα, or Ziβ or Wiβ,
or directed towards the exterior of said compounds, and are respectively designated Ze and We.

3. Compounds according to claim 1 [[or 2]], characterized in that A, B, C, and D are in ortho position, and/or in that E represents in combination with F, and H represents in combination with G, CH₂—CH₂.

4. Compounds according to claim 1, characterized in that A forms with C, and B forms with D, chain formations of formula (1) respectively designated A-C and B-D, these two chain formations being situated in α position.

5. Compounds according to claim 4, characterized in that:
the A-C and B-D chain formations each comprise a Ziα group and a We group,
or the A-C chain formation comprises a Ziα group and a We group, whilst the B-D chain formation comprises a Ze group and a Wiα group,
or the A-C and B-D chain formations each comprise a Ze group and a Wiα group.

6. Compounds according to claim 4 characterized by the following formulae:

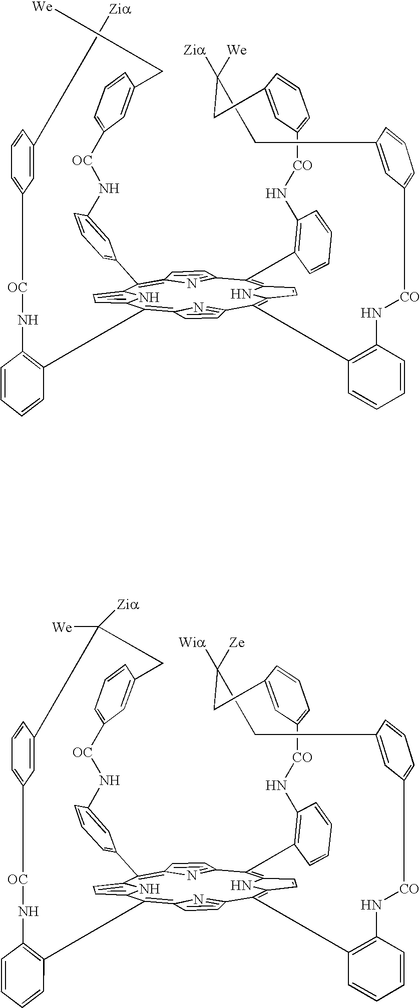

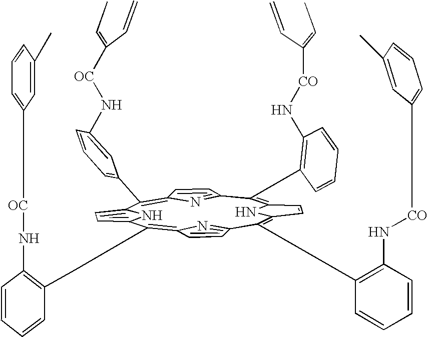

7. Compounds according to claim 1, characterized in that A forms with C an A-C chain formation of formula (1) situated in α position, and B forms with D, a B-D chain formation of formula (1) situated in β position.

8. Compounds according to claim 7, characterized in that:
the A-C chain formation comprises a Ziα group and a We group, whilst the B-D chain formation comprises a Ziβ group and a We group,
or the A-C chain formation comprises a Ze group and a Wiα group, whilst the B-D chain formation comprises a Ziβ group and a We group,
or the A-C chain formation comprises a Ze group and a Wiα group, whilst the B-D chain formation comprises a Ze group and a Wiβ group.

9. Compounds according to claim 7 [[or 8]], characterized by the following formulae:

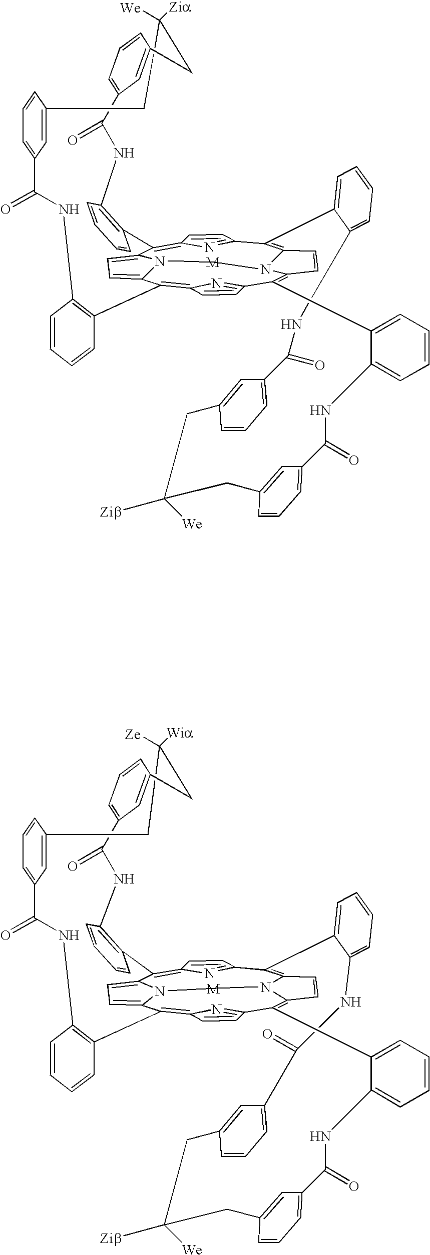

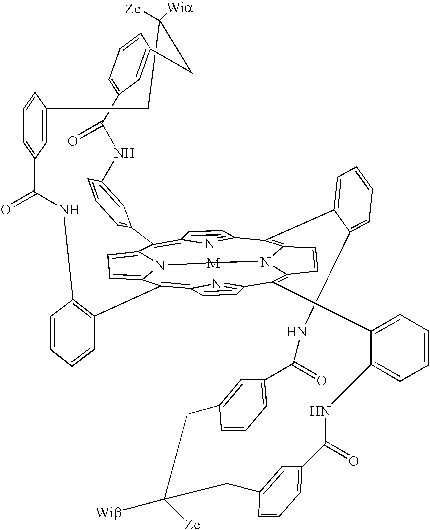

10. Compounds according to claim 1, characterized in that A forms with D an A-D chain formation of formula (1) situated in β position, and B forms with C, a B-C chain formation of formula (1) situated in α position.

11. Compounds according to claim 10, characterized in that:
the A-D chain formation comprises a Ze group and a Wiβ group, whilst the B-C chain formation comprises a Ze group and a Wiα group,
or the A-D chain formation comprises a Ziβ group and a We group, whilst the B-C chain formation comprises a Ze group and a Wiα group,
or the A-D and B-C chain formations each comprise a Ziβ group and a We group.

12. Compounds according to claim 10 characterized by the following formulae:

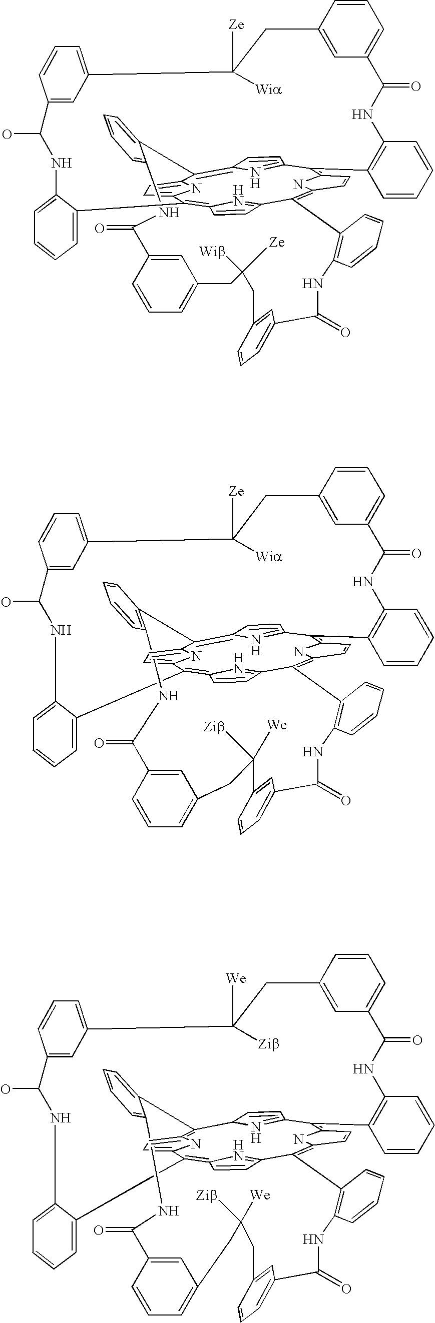

13. Complexes between a compound according to claim 1, and a radioelement chosen from the α emitters, or a divalent or trivalent metallic element.

14. Complexes between a compound according to claim 1, and an α-emitter radioelement chosen from bismuth-212 or -213, actinium-225, or astatine-211.

15. Complexes between a compound according to claim 1, and a divalent or trivalent metallic element chosen from Y(III), In(III), Cd(II), Mg(II), Mn(III), Fe(III), B(III) and the lanthanides.

16. Pharmaceutical composition characterized in that it comprises a complex according to claim 13, in combination with a pharmaceutically acceptable vehicle.

17. Pharmaceutical composition according to claim 16, characterized in that it is presented in a form which can be administered by intravenous route.

18. Use of complexes defined in claim 13, for preparing a medicament intended for the treatment of cancer, or for preparing compositions intended for medical imaging.

19. Use according to claim 18, for preparing a medicament intended for the treatment of tumorous small-cell cancers, such as acute myeloid leukemia, non-Hodgkin's lymphomas, bronchopulmonary dysplasias, metastatic breast cancers, colorectal cancers, lymphomas, and pathologies in which the following antigenic units are involved: CD52, CD22, CD20, HLA-DR, CD33, LE-Y, Ep-CAM, ACE, CAN, EGFR, KSA, VEGF, HER2, GD2, tenascin.
